(19) United States
(12) Patent Application Publication
(10) Pub. No.: US 2026/0356787 A1
(43) Pub. Date: Nov. 12, 2026

(54) NOVEL EFFICIENT METHOD FOR THE RECOVERY OF ACRYLIC VALUE FROM ACRYLIC ACID OLIGOMERS

(71) Applicant: Rohm and Haas Company, Collegeville, PA (US)

(72) Inventors: Reetam Chakrabarti, Phoenixville, PA (US); Joseph F. Dewilde, King of Prussia, PA (US); James E. Elder, Friendswood, TX (US); Lan T.P. Hoang Nguyen, Doylestown, PA (US); Muhunthan Sathiosatham, Chalfont, PA (US); Mark A. Silvano, Upper Black Eddy, PA (US)

(21) Appl. No.: 19/391,347

(22) Filed: Nov. 17, 2025

Related U.S. Application Data
(60) Provisional application No. 63/728,470, filed on Dec. 5, 2024.

(51) Int. Cl. C07C 57/04; C07C 51/09; C07C 51/44; C07C 67/08; C07C 69/54

(52) U.S. Cl. CPC C07C 57/04; C07C 51/09; C07C 51/44; C07C 67/08; C07C 69/54

(57) ABSTRACT

A process for recovering acrylic values from Michael addition products comprises reacting a liquid stream containing acrylic acid and Michael addition products of acrylic acid with a C1 to C8 alkyl alcohol in the presence of an esterification or transesterification catalyst and heating to a temperature ranging from 100 to 135 °C to form an intermediate mixture comprising a C1 to C8 alkyl acrylate and a C1 to C8 alkyl-3-hydroxy propionate and compounds of formula (VI)

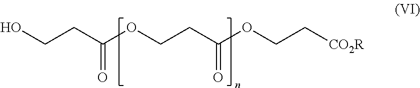

wherein R is a C1 to C8 alkyl group and n ranges from 1 to 3. The C1 to C8 alkyl-3-hydroxy propionate, the compounds of formula (VI) and any remaining Michael addition products of acrylic acid or C1 to C8 alkyl esters thereof present in the intermediate mixture are thermally cracked to form a product comprising acrylic acid and C1 to C8 alkyl acrylate.

### FIELD OF THE INVENTION

The invention relates to a method for recovering acrylic value from acrylic acid oligomers.

### BACKGROUND OF THE INVENTION

Acrylic acid and its ester derivatives are used in the manufacture of a broad spectrum of polymer products found across multiple market segments. For example, poly-acrylic acid (PAA) formulations are commonly found in detergent applications, sodium salts of PAA are used as super absorbent polymers (SAP) found in baby diapers and other adult hygiene products. Copolymers of acrylic monomers with methacrylic monomers, styrene, butadiene and other functionalized vinyl monomers are used to manufacture dispersions and resins that are ubiquitous in consumer and industrial products such as architectural, industrial, paper and automotive coatings, performance additives for concrete admixtures and asphalts, textiles, adhesives, sealants and caulks to name just a few applications.

Commercially, acrylic monomer production is dominated by acrylic acid which serves as a platform chemical for a broad range of acrylate derivatives. Acrylic acid may be produced using a variety of different methods. For example, acrylic acid can be made via propylene oxidation, bio-based processes (e.g., using any one of glycerol, 3-hydroxypropionic acid, lactate, lactic acid, etc., as a starting material), the ethylene cyanohydrin process, the acetylene process (i.e., the Reppe process), the β-propiolactone process, acrylonitrile hydrolysis, or coupling carbon dioxide with ethylene or ethane. Industrially, most of the global capacity of acrylic acid is manufactured via the two-stage heterogeneously catalyzed oxidation of propylene over mixed metal oxide catalysts.

The crude acrylic acid (CAA) that is produced by any of the known methods contains Michael addition products, which include dimers, trimers, and higher-mers of acrylic acid. These Michael addition products may reversibly react to form acrylic acid or higher or lower oligomers.

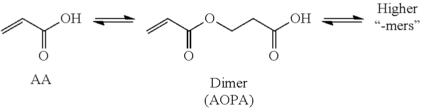

Michael addition products of acrylic acids are also present in waste streams and bottoms streams from acrylic acid purification processes, as well as waste and bottoms streams from ester production processes that use acrylic acid in the formation of the esters, such as, for example, butyl acrylate production.

Dimer crackers are typically used to recover acrylic acid value from the Michael addition products present in these streams.

Attempts have been made to improve acrylic acid recovery in dimer crackers. Some methods utilize strong acids as a catalyst to facilitate recovery of acrylic value.

European Patent EP 1275633 discloses a process for decomposing Michael adducts of acrylic acid by reacting in the presence of an N-oxyl compound to form acrylic acid, acrylic ester, and/or alcohol.

U.S. Pat. No. 7,612,231 discloses a process for recovering acrylic acid from a mixture comprising acrylic acid dimer by carrying out decomposition of the acrylic acid dimer under reduced pressure while recovering acrylic acid by distillation in a process having a retention time at the bottom of the acrylic acid recovering device of 10 to 30 hours.

U.S. Pat. No. 8,308,913 discloses an apparatus for recovering acrylic acid comprising an acrylic acid recovering device having an acrylic acid recovery device having a thin film evaporator integrated with an acrylic acid dimer pyrolysis tank.

U.S. Pat. No. 3,868,410 discloses an esterification process wherein oligomeric byproducts are converted to acrylic acid, an alkanol, or a alkylate of the alkanol by heating the oligomeric byproducts at a temperature of at least 180 °C.

The methods disclosed in the references cited above recover at most only 50 wt % of eligible acrylic acid molar equivalents.

There is a need in the art for improved recovery of acrylic acid from Michael addition products.

### SUMMARY OF THE INVENTION

The invention is directed to a process for recovering acrylic values from Michael addition products comprising:
a) reacting a liquid stream comprising acrylic acid and Michael addition products of acrylic acid with a C1 to C8 alkyl alcohol in the presence of an esterification or transesterification catalyst and heating to a temperature ranging from 80 to 135 °C to form an intermediate mixture comprising a C1 to C8 alkyl acrylate and a C1 to C8 alkyl-3-hydroxy propionate and compounds of formula (VI):

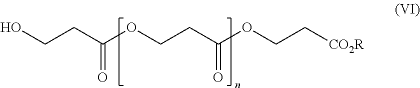

wherein R is a C1 to C8 alkyl group and n ranges from 1 to 7; and
b) thermally cracking the C1 to C8 alkyl-3-hydroxy propionate, the compounds of formula (VI) and any remaining Michael addition products of acrylic acid or C1 to C8 alkyl esters thereof present in the intermediate mixture to form a product comprising acrylic acid and C1 to C8 alkyl acrylate.

### BRIEF DESCRIPTION OF THE DRAWINGS

FIG. 1 is a diagram the reaction of a first step in the process according to an embodiment of the present invention.

FIG. 2 is a diagram of a final step in the process according to an embodiment of the present invention.

### DETAILED DESCRIPTION OF THE INVENTION

All percentage compositions are weight percentages (wt %), and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system.

As used herein, the terms “Michael addition products of acrylic acid” and “dimer, trimer, and higher-mers of acrylic acid” refer to oligomers of acrylic acid. The oligomers of acrylic acid can reversibly react to form higher or lower oligomers, as well as acrylic acid.

The present invention relates to a process for recovering acrylic value from heavy waste byproducts, such as Michael addition products of acrylic acid. As used herein “acrylic value” refers to acrylic acid and esters of acrylic acid that are useful in the production of polymers.

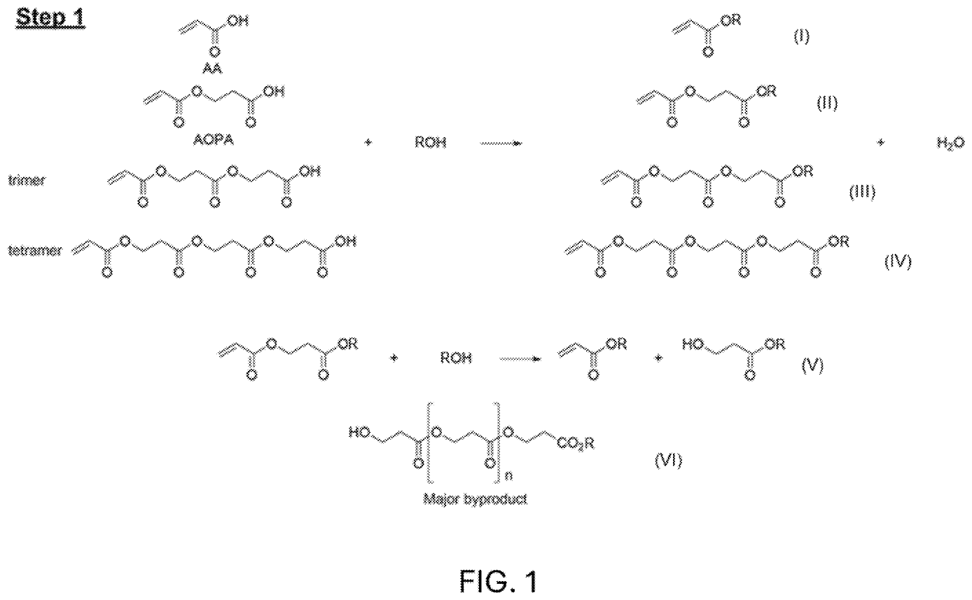

In the inventive process, acrylic value recovery is performed by reacting a liquid stream comprising acrylic acid and Michael addition products of acrylic acid with a C1 to C8 alkyl alcohol (i.e., ROH, where R is a C1 to C8 alkyl group) to form an intermediate mixture as shown in FIG. 1. The liquid stream comprises acrylic acid, the dimer of acrylic acid (3-(acryloyloxy) propanoic acid or AOPA), acrylic acid trimer, and acrylic acid tetramer. Higher-mers of acrylic acid may also be present. The dimer is the majority component of the Michael addition products. More preferably, the dimer is the majority component in the liquid stream comprising acrylic acid and its Michael addition products.

When reacted with a C1 to C8 alkyl alcohol, C1 to C8 alkyl esters of the acrylic acid (i.e., C1 to C8 alkyl acrylates) and its Michael addition products (compounds of formulae (I) to (IV) in FIG. 1) are formed.

The alkoxy-3-acryloyloxypropanoic acid of formula (II) further reacts with the C1 to C8 alkyl alcohol to form an alkyl-3-hydroxy propionate of formula (V) and, as a major byproduct, oligomers thereof (compounds of formula (VI)).

Thus, the first step yields an intermediate stream mixture comprising compounds of formulae (I) to (VI).

The alkyl-3-hydroxy propionate of formula (V) and its oligomers (compounds of formula (VI)) are generally regarded as an undesirable byproducts. However, the present inventors have discovered that acrylic value recovery can be improved by driving the reaction to convert the Michael addition products to form compounds of formulae (V) and (VI) and then thermally cracking to recover acrylic value. Significantly higher acrylic value recoveries can be achieved compared to traditional dimer cracker processes.

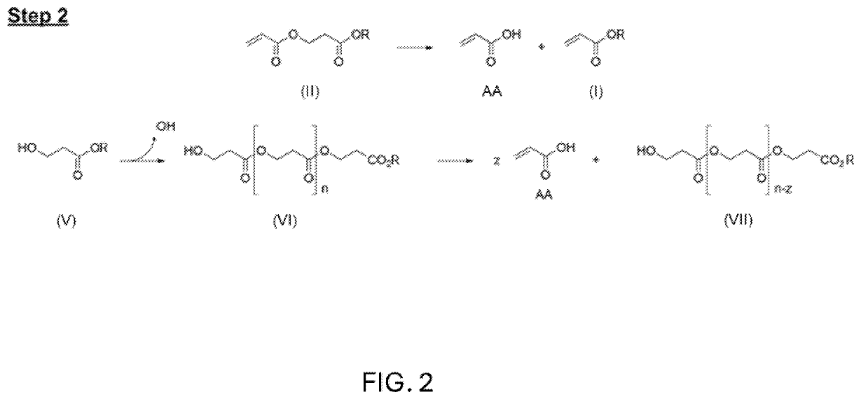

In a subsequent step (FIG. 2), the intermediate reaction mixture is thermally cracked to form acrylic acid and C1 to C8 alkyl acrylates. In the thermal cracking reaction, alkoxy-3-acryloyloxypropanoic acid of formula (II) forms acrylic acid and a C1 to C8 alkyl acrylate. The higher oligomeric compounds of formulae (III) and (IV) are thermally cracked to form acrylic acid and lower oligomers, which are further reduced to form acrylic acid and the C1 to C8 alkyl acrylate. The compounds of formulae (V) and (VI) are thermally cracked to form acrylic acid and lower oligomers of formula (VI), which further react to form acrylic acid and the corresponding C1 to C8 alkyl acrylate.

The C1 to C8 alkyl alcohol preferably comprises methanol, ethanol, butanol, and 2-ethyl hexyl alcohol. More preferably, the C1 to C8 alkyl alcohol is butanol.

When the C1 to C8 alkyl alcohol is butanol, the first step of the process forms an intermediate mixture comprising butyl acrylate (compound (I) where R is a butyl group), butyoxy-3-acryloyloxypropanoic acid (BAOPA; compound (II) where R is a butyl group), and higher oligomers (compounds (III) and (IV) where R is a butyl group).

BAOPA (compound (II) where R is a butyl group) further reacts with butanol to form butyl acrylate and butyl-3-hydroxy propionate (BBHP; compound (V) where R is a butyl group). Higher oligomers of BBHP (compounds of formula (VI)) are also formed as a major byproduct.

In a subsequent step, the intermediate mixture comprising compounds (II) to (VI) where R is a butyl group is thermally cracked. The BAOPA is reduced to acrylic acid and butyl acrylate. The BBHP and higher oligomers thereof form acrylic acid and lower oligomers of BBHP, which further react to recover additional acrylic value.

The first step of the process is conducted in the presence of an esterification or transesterification catalyst. Esterification and transesterification catalysts include, for example, ion exchange resins and strong acids, such as hydrochloric acid, sulfuric acid, p-toluenesulfonic acid, phosphoric acid, and methanesulfonic acid. Preferably, the catalyst is a strong acid, more preferably sulfuric acid.

A polymerization inhibitor is added during the first step, the thermal cracking step, or both. Alternatively, a polymerization inhibitor may already be present in the liquid stream used in the inventive process by virtue of being added during an upstream processing step. Any suitable polymerization inhibitor known in the art may be used in the process. Examples of preferred inhibitors include soluble manganese ions, soluble copper ions, hydroquinone (HQ), monomethyl ether hydroquinone (MEHQ), phenothiazine (PTZ), 2,2,6,6-tetramethyl-1-piperidinyloxy (TEMPO) and related compounds such as 4-hydroxy TEMPO. Combinations of inhibitors can be employed, such as, for example, a mixture of a source of soluble manganese ions, such as manganese acetate, and 4-hydroxy TEMPO. Molecular oxygen or air may also be used, as oxygen is known to be an inhibitor. The inhibitor is employed in an amount sufficient to prevent or reduce the polymerization of acrylic acid, as is well known to those skilled in the art.

The first step is conducted at a temperature ranging from 80 to 135 °C, preferably from 90 to 135 °C, more preferably from 100 to 135 °C, and still more preferably from 110 to 135 °C.

The thermal cracking step is preferably conducted at a temperature of at least 135 °C, more preferably 150 °C, and even more preferably at least 175 °C.

Acrylic values may be removed at any step of the process. For example, acrylic acid and/or the C1 to C8 alkyl acrylates may be removed as a vapor stream or distillate from the reaction of the first step.

A separation step may be conducted between the first step and the thermal cracking step. For example, the intermediate stream may be subjected to distillation to recover additional acrylic values before the thermal cracking step. If present, the distillation step is preferably carried out at a temperature ranging from 40 to 80 °C, preferably from 45 to 70 °C, and at an absolute pressure less than 70 mmHg, more preferably less than 40 mmHg, and even more preferably less than 10 mmHg, and most preferably approaching 0 mmHg.

The present invention surprisingly enables the recovery of a significant amount of the Michael addition products of acrylic acid from the feed. For example, when butanol is used as the C1 to C8 alkyl alcohol, approximately 85 to 90 wt % of the Michael addition products of acrylic acid are recovered as acrylic acid or butyl acrylate. This represents a significant improvement over conventional dimer cracking methods.

Preferably, the liquid stream used in the present invention comprises acrylic acid generated in any known acrylic acid production process. For example, acrylic acid can be made via propylene oxidation, a bio-based process (e.g., using any one of glycerol, 3-hydroxypropionic acid, lactate, lactic acid, etc., as a starting material), an ethylene cyanohydrin process, an acetylene process (i.e., the Reppe process), a β-propiolactone process, an acrylonitrile hydrolysis process, or a process performed by coupling carbon dioxide with ethylene or ethane. The reaction mixture produced during the acrylic acid production process may be processed in one or more unit operations. These operations may include, for example, cooling processes, quenching processes, separations, purifications, etc., or a downstream reaction such as an ester production process.

Preferably, the liquid stream used in the inventive process comprises a low grade acrylic acid stream, a waste stream, or a bottoms stream from an acrylic acid purification process that draws off a product stream that is substantially comprised of acrylic acid, such as, for example, a product stream comprising at least 90 wt % acrylic acid. The liquid stream also, or alternatively, comprises a waste stream or a bottoms stream from an ester production process, preferably an alkyl acrylate production process. The low grade acrylic acid stream, waste stream, or bottoms stream comprises Micheal addition products of acrylic acid and at least some amount of acrylic acid from the acrylic acid production process and is reacted with the C1 to C8 alkyl alcohol to recover acrylic values.

### EXAMPLES

### Example 1

### Step 1—Esterification

A 1000 ml 4-neck round-bottom flask equipped with a magnetic stirrer, thermocouple, and a Dean-Stark distillation head was charged with AOPA (250 g; Aldrich containing 19.4% acrylic acid, 32.9% AOPA, 21.4% trimer, and 21.4% tetramer), BuOH (300 g, 4.05 mol), sulfuric acid (15 g, 0.15 mol), and PTZ (1 g). The resulting solution was mixed well and a sample was taken for NMR analysis. The contents of the flask were slowly heated to 135 °C and the distillate was collected in the Dean-Stark apparatus. The distillation commenced when the pot temperature was at 105 °C and the vapor temperature was between 95 °C and 100 °C. The total amount of water collected was 34 g and the organic distillate (D1) weighed 18 g and was analyzed by quantitative NMR and gas chromatography (GC). The heating was turned off when the distillation ceased and the pot contents were allowed to cool to room temperature. The soluble polymers in the reaction mixture were filtered off using a Buchner funnel and weighed 8 g upon air drying. Filtrate weighed 498 g.

### Step 2—Distillation

The filtrate from the above step was transferred back to the round-bottom flask and the Dean-Stark head was replaced with a short-path distillation head and the pressure was reduced to <1 mmHg absolute. The contents of the flask were heated to 60 °C and the distillate was collected at the vapor temperature range of 33 °C to 38 °C. This distillation was done to remove excess BuOH and product BA before the thermal cracking step. The heating turned off and the vacuum was released when the vapor temperature dropped to below 25 °C. The distillate (D2) collected weighed 247 g and was analyzed by quantitative NMR and GC.

### Step 3—Thermal Cracking

The setpoint was increased to 180 °C and the pressure was reduced to 100 mmHg. The distillate began to collect when the pot temperature was at 135 °C and continue to collect for 60 minutes at the vapor temperature range of 100 °C to 135 °C. After 60 minutes distillate collection ceased and the amount of distillate (D3) collected was 242 g. Pot residue, black solid, weighed 7.5 g.

Heavies recovery was 87%. The results for Experiment 1 are shown below in Table 1.

TABLE 1

| | molar | mol % |
|---|---|---|
| Overall C3 yield (AA + BA) | 1.95 | 90% |
| Heavies recovery | 0.37 | 87% |
| Overall C4 yield (BA + BuOH) | 2.73 | 89% |

### Example 2

### Step 1—Esterification

A 1000 ml 4-neck round-bottom flask equipped with a magnetic stirrer, thermocouple, and a Dean-Stark distillation head was charged with the bottoms from a glacial acrylic acid (GAA) production process (250 g), BuOH (300 g, 4.05 mol), sulfuric acid (15 g, 0.15 mol), and PTZ (1 g). The bottoms of the GAA production process contained 45.7% acrylic acid, 33.4% AOPA, and 8.0% trimer. The resulting solution was mixed well and a sample was taken for NMR analysis. The contents of the flask were slowly heated to 135 °C and the distillate was collected in the Dean-Stark apparatus. The distillation commenced when the pot temperature was at 107.5 °C and the vapor temperature was between 98 °C and 110 °C. The total amount of water collected was 45 g and the organic distillate (D1) weighed 23 g and was analyzed by quantitative NMR and GC.

### Step 2—Distillation

The product mix from the above step was subjected to distillation. The Dean-Stark head was replaced with a short-path distillation head and the pressure was reduced to <1 mmHg absolute. The contents of the flask were heated to 60 °C and the distillate was collected at the vapor temperature range of 30 °C to 35 °C. This distillation was done to remove excess BuOH and product BA before the thermal cracking step. The heating turned off and the vacuum was released when the vapor temperature dropped to below 25 °C. The distillate (D2) collected weighed 267 g and was analyzed by quantitative NMR and GC.

### Step 3—Thermal Cracking

The setpoint was increased to 180 °C and the pressure was reduced to 200 mmHg. The distillate began to collect when the pot temperature was at 145 °C and continue to collect for 40 minutes at the vapor temperature range of 85 °C to 135 °C. After 60 minutes distillate collection ceased and the amount of distillate (D3) collected was 131 g. Pot residue, black solid, weighed 55 g.

Heavies recovery was 74%. The results of Example 2 are shown below in Table 2.

TABLE 2

| | molar | mol % |
|---|---|---|
| Overall C3 yield (AA + BA) | 2.64 | 88% |
| Heavies recovery | 1.05 | 74% |
| Overall C4 yield (BA + BuOH) | 3.13 | 89% |

### Example 3

### Step 1—Esterification

A 1000 ml 4-neck round-bottom flask equipped with a magnetic stirrer, thermocouple, and a Dean-Stark distillation head was charged with a GAA bottoms stream (250 g), BuOH (300 g, 4.05 mol), sulfuric acid (15 g, 0.15 mol), and PTZ (1 g). The GAA bottoms stream contained 45.7% acrylic acid, 33.4% AOPA, and 8.0% trimer. The resulting solution was mixed well and a sample was taken for NMR analysis. The contents of the flask were slowly heated to 135 °C and the distillate was collected in the Dean-Stark apparatus. The distillation commenced when the pot temperature was at 107.5 °C and the vapor temperature was between 98 °C and 110 °C. The total amount of water collected was 46 g and the organic distillate (D1) weighed 10 g and was analyzed by quantitative NMR and GC.

### Step 2—Distillation

The product mix from the above step was subjected to distillation. The Dean-Stark head was replaced with a short-path distillation head and the pressure was reduced to 70 mmHg. The contents of the flask were heated to 115 °C and the distillate was collected at the vapor temperature range of 60 °C to 80 °C. This distillation was done to remove excess BuOH and product BA before the thermal cracking step. The heating turned off and the vacuum was released when the vapor temperature dropped to below 25 °C. The distillate (D2) collected weighed 270 g and was analyzed by quantitative NMR and GC.

### Step 3—Thermal Cracking

The setpoint was increased to 180 °C and the pressure was reduced to 200 mmHg. The distillate began to collect when the pot temperature was at 145 °C and continue to collect for 40 minutes at the vapor temperature range of 85 °C to 125 °C. After 60 minutes distillate collection ceased and the amount of distillate (D3) collected was 110 g. Pot residue, black solid, weighed 85 g.

Heavies recovery was 55%. The results of Example 3 are shown below in Table 3. The reduced heavies recovery was believed to be the result of the higher temperature and pressure in the distillation step.

TABLE 3

| | molar | mol % |
|---|---|---|
| Overall C3 yield (AA + BA) | 2.37 | 78% |
| Heavies recovery | 0.79 | 55% |
| Overall C4 yield (BA + BuOH) | 2.97 | 85% |

### Example 4

### Step 1—Esterification

A 1000 ml 4-neck round-bottom flask equipped with a magnetic stirrer, thermocouple, and a Dean-Stark distillation head was charged with GAA bottoms stream (250 g), BuOH (300 g, 4.05 mol), sulfuric acid (22.5 g, 0.23 mol), and PTZ (1 g). The GAA bottoms stream contained 45.7% acrylic acid, 33.4% AOPA, and 8.0% trimer. The resulting solution was mixed well and a sample was taken for NMR analysis. The contents of the flask were slowly heated to 135 °C and the distillate was collected in the Dean-Stark apparatus. The distillation commenced when the pot temperature was at 107.5 °C and the vapor temperature was between 98 °C and 115 °C. The total amount of water collected was 46 g and the organic distillate (D1) weighed 9.5 g and was analyzed by quantitative NMR and GC.

### Step 2—Distillation

The product mix from the above step was subjected to distillation. The Dean-Stark head was replaced with a short-path distillation head and the pressure was reduced to <1 mmHg absolute. The contents of the flask were heated to 60 °C and the distillate was collected at the vapor temperature range of 35 °C to 45 °C. This distillation was done to remove excess BuOH and product BA before the thermal cracking step. The heating turned off and the vacuum was released when the vapor temperature dropped to below 25 °C. The distillate (D2) collected weighed 270 g and was analyzed by quantitative NMR and GC.

### Step 3—Thermal Cracking

The setpoint was increased to 180 °C and the pressure was reduced to 200 mmHg. The distillate began to collect when the pot temperature was at 145 °C and continue to collect for 40 minutes at the vapor temperature range of 85 °C to 125 °C. After 60 minutes distillate collection ceased and the amount of distillate (D3) collected was 155 g. Pot residue, black solid, weighed 85 g.

Heavies recovery was 90%. The results from Example 4 are shown below in Table 4.

TABLE 4

| | molar | mol % |
|---|---|---|
| Overall C3 yield (AA + BA) | 2.87 | 95% |
| Heavies recovery | 1.29 | 90% |
| Overall C4 yield (BA + BuOH) | 3.13 | 89% |

1. A process for recovering acrylic values from Michael addition products comprising:
a) reacting a liquid stream comprising acrylic acid and Michael addition products of acrylic acid with a C1 to C8 alkyl alcohol in the presence of an esterification or transesterification catalyst and heating to a temperature ranging from 80 to 135 °C to form an intermediate mixture comprising a C1 to C8 alkyl acrylate and a C1 to C8 alkyl-3-hydroxy propionate and compounds of formula (VI): 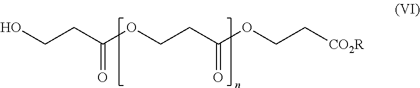
wherein R is a C1 to C8 alkyl group and n ranges from 1 to 7; and
b) thermally cracking the C1 to C8 alkyl-3-hydroxy propionate, the compounds of formula (VI) and any remaining Michael addition products of acrylic acid or C1 to C8 alkyl esters thereof present in the intermediate mixture to form a product comprising acrylic acid and C1 to C8 alkyl acrylate.

2. The process of claim 1, wherein the C1 to C8 alkyl alcohol is selected from the group consisting of methanol, ethanol, butanol, and 2-ethyl hexyl alcohol.

3. The process of claim 2, wherein the C1 to C8 alkyl alcohol is butanol.

4. The process of claim 1, further comprising removing C1 to C8 alkyl acrylate and/or C1 to C8 alkyl alcohol from the intermediate mixture before the thermal cracking step.

5. The process of claim 4, wherein the C1 to C8 alkyl acrylate and/or C1 to C8 alkyl alcohol are removed from the intermediate mixture by distillation.

6. The process of claim 1, wherein thermally cracking the C1 to C8 alkyl-3-hydroxy propionate and the compounds of formula (VI) and any remaining Michael addition products of acrylic acid or C1 to C8 alkyl esters thereof present in the intermediate mixture comprises heating the C1 to C8 alkyl-3-hydroxy propionate and the compounds of formula (VI) to a temperature at least 135 °C.

7. The process of claim 6, wherein thermally cracking the C1 to C8 alkyl-3-hydroxy propionate, the compounds of formula (VI) and any remaining Michael addition products of acrylic acid or C1 to C8 alkyl esters thereof present in the intermediate mixture comprises heating the C1 to C8 alkyl-3-hydroxy propionate and the compounds of formula (VI) to a temperature at least 150 °C.

8. The process of claim 7, wherein thermally cracking the C1 to C8 alkyl-3-hydroxy propionate and the compounds of formula (VI) and any remaining Michael addition products of acrylic acid or C1 to C8 alkyl esters thereof present in the intermediate mixture comprises heating the C1 to C8 alkyl-3-hydroxy propionate and the compounds of formula (VI) to a temperature at least 175 °C.

9. The process of claim 1, wherein the acrylic acid in the liquid stream comprising acrylic acid is produced by a method selected from the group consisting of propylene oxidation-based process, a bio-based process, an ethylene cyanohydrin-based process, an acetylene-based process, a β-propiolactone-based process, an acrylonitrile hydrolysis-based process, or a process performed by coupling carbon dioxide with ethylene or ethane.
